(19) United States
(12) Patent Application Publication
(10) Pub. No.: US 2022/0122689 A1
(43) Pub. Date: Apr. 21, 2022

(54) SYSTEMS AND METHODS FOR ALIGNMENT-BASED PRE-TRAINING OF PROTEIN PREDICTION MODELS

(71) Applicant: Salesforce.com, Inc., San Francisco, CA (US)

(72) Inventors: Pascal Sturmfels, Seattle, WA (US); Ali Madani, San Francisco, CA (US); Jesse Vig, Los Altos, CA (US); Nazneen Rajani, Mountain View, CA (US)

(21) Appl. No.: 17/153,164

(22) Filed: Jan. 20, 2021

Related U.S. Application Data
(60) Provisional application No. 63/092,223, filed on Oct. 15, 2020.

(51) Int. Cl. G16B 5/20; G16B 30/10; G16B 40/20

(52) U.S. Cl. CPC G16B 5/20; G16B 40/20; G16B 30/10

(57) ABSTRACT

Embodiments described herein provide an alignment-based pre-training mechanism for protein prediction. Specifically, the protein prediction model takes as input features derived from multiple sequence alignments (MSAs), which cluster proteins with related sequences. Features derived from MSAs, such as position specific scoring matrices and hidden Markov model (HMM) profiles, have long known to be useful features for predicting the structure of a protein. Thus, in order to predict profiles derived from MSAs from a single protein in the alignment, the neural network learns information about that protein's structure using HMM profiles derived from MSAs as labels during pre-training (rather than as input features in a downstream task).

### CROSS REFERENCE(S)

The present disclosure is a nonprovisional of and claims priority under 35 U.S.C. 119 to U.S. provisional application No. 63/092,223, filed Oct. 15, 2020, which is hereby expressly incorporated by reference herein in its entirety.

### TECHNICAL FIELD

The present disclosure relates generally to machine learning models and neural networks, and more specifically, to an alignment-based pre-training of protein prediction models.

### BACKGROUND

Artificial intelligence, implemented with neural networks and deep learning models, has demonstrated great promise as a technique for automatically analyzing real-world information with human-like accuracy. Recently a potential application for artificial intelligence has been adopted in the field of protein engineering for using a machine learning model to predict the properties of a specific protein sequence. Traditionally, experimentally determining properties about protein sequences, such as structure or intrinsic stability, is usually expensive. Predicting these properties directly from protein sequences using machine learning models is of great interest, as it could speed up downstream biological discovery. However, for protein sequence datasets, unlabeled data has greatly outpaced labeled data due to the high cost of wet-lab characterization.

Therefore, there is a need to utilize unlabeled protein sequence data to pre-train a protein prediction model.

### BRIEF DESCRIPTION OF THE DRAWINGS

FIG. 1 is a simplified block diagram 100 illustrating an overview of alignment-based pre-training of a language model for protein sequencing, according to embodiments described herein.

FIG. 2 illustrates an overview process of the proposed task of generating labels from hidden Markov model profiles during pre-training, according to embodiments described herein.

FIG. 3 is a simplified diagram of a computing device that implements and pre-trains a protein sequence model, according to some embodiments.

FIG. 4 is a simplified logic flow diagram illustrating a method for pre-training a transformer network for protein profile prediction, according to some embodiments described herein.

FIGS. 5-8 provide performance charts illustrating performance of the pre-training task described in FIGS. 1-4 compared against existing systems, according to some embodiments described herein.

In the figures and appendix, elements having the same designations have the same or similar functions.

### DETAILED DESCRIPTION

Traditionally, experimentally determining properties about protein sequences, such as structure or intrinsic stability, is usually expensive. Predicting these properties directly from protein sequences using machine learning models is of great interest, as it could speed up downstream biological discovery. However, for protein sequence datasets, unlabeled data has greatly outpaced labeled data due to the high cost of wet-lab characterization. Some existing computer vision or natural language processing (NLP) models leverage large, unlabeled datasets via self-supervised pre-training, e.g., training a machine learning model using a loss function derived solely from the unlabeled data.

Recent research results have observed several similarities between protein sequence modeling and NLP—namely, sequences comprised of a discrete set of characters as input, and far more unlabeled data than labeled. Some existing systems adapt NLP models to protein sequence tasks, including pre-training tasks, namely, masked language modeling and auto regressive generation. Unfortunately, on some tasks such as secondary structure and contact prediction, the existing pre-training has compromised performance which often fails to capture the underlying protein biology.

In view of the need for a pre-training mechanism for protein sequence models with unlabeled data, embodiments described herein provide an alignment-based pre-training mechanism for protein prediction. Specifically, the protein prediction model takes as input features derived from multiple sequence alignments (MSAs), which cluster proteins with related sequences. Features derived from MSAs, such as position specific scoring matrices and hidden Markov model (HMM) profiles, have long known to be useful features for predicting the structure of a protein. Thus, in order to predict profiles derived from MSAs from a single protein in the alignment, the neural network learns information about that protein's structure using HMM profiles derived from MSAs as labels during pre-training (rather than as input features in a downstream task).

As used herein, the term “network” may comprise any hardware or software-based framework that includes any artificial intelligence network or system, neural network or system and/or any training or learning models implemented thereon or therewith.

As used herein, the term “module” may comprise hardware or software-based framework that performs one or more functions. In some embodiments, the module may be implemented on one or more neural networks.

### Overview

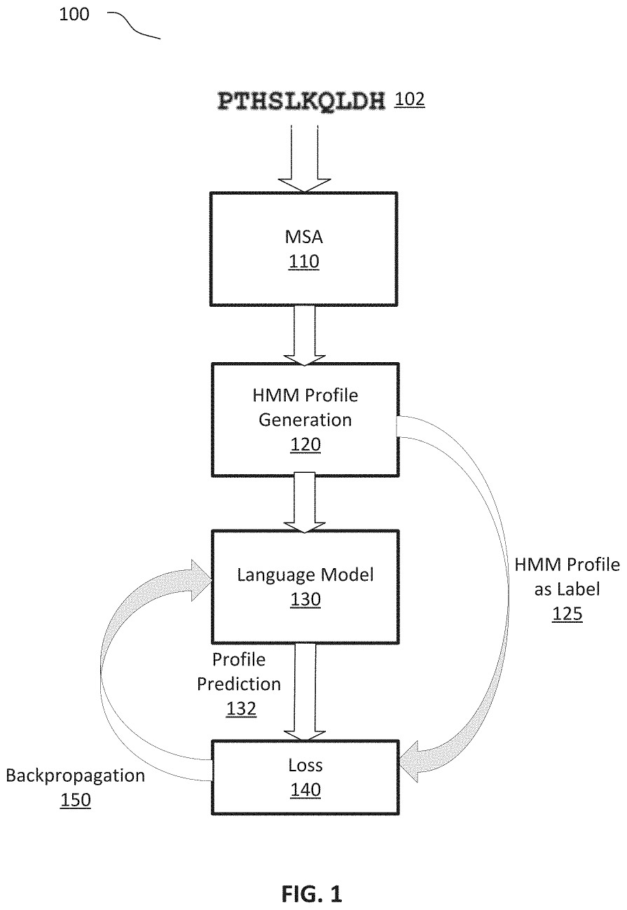

FIG. 1 is a simplified block diagram 100 illustrating an overview of alignment-based pre-training of a language model for protein sequencing, according to embodiments described herein. Diagram 100 shows that input data sequences 102 representing the amino acid sequences that form certain proteins are used to pre-train a language model 130, such as a Transformer network, for protein sequencing, e.g., to predict the profile properties of a protein based on an input data sequence of amino acids.

For example, the pre-training data of input data sequences 102 may be an unlabeled protein sequence from associated data sets for a set of five standardized protein sequence prediction tasks plus a large unlabeled pre-training dataset derived from Pfam in Rao et al., Evaluating protein transfer learning with tape, in Advances in Neural Information Processing Systems, pages 9689-9701, 2019, which is hereby expressly incorporated by reference herein in its entirety.

Input data sequences 102 may be passed to a multiple sequence alignment (MSA) module 110, which clusters related data sequences together as proteins that belong to the same family. The MSA module 110 may derive features from the clustered data sequences, such as in the form of position-specific scoring matrices. Specifically, MSA module 110 arranges proteins in a matrix whose rows are individual protein sequences and whose columns contain amino acids that either come from the same position in some ancestral sequence (homologous), or play a common structural or functional role. For example, the pre-training data sequences 102 may be similar to the MSA pre-training data introduced in Rao et al. The pre-training data set may comprise 32 million sequences from Pfam, which further contains pre-built MSAs for each of its entries, grouped into a set of families. In one embodiment, the MSA module 110 uses the existing multiple sequence alignments from the 32.0 release of Pfam. In another embodiment, the MSA module 110 may build a set of multiple sequence alignments for any protein sequence dataset using standard alignment tools.

For example, for the input data sequence x=(x₁, x₂, . . . xₙ) representing a protein sequence of length n, the MSA group that the input sequence belongs to is represented by an MSA matrix:

$$A = ( \begin{matrix}{a}_{11} & {a}_{12} & … & {a}_{1 m} \\ {a}_{21} & {a}_{22} & … & {a}_{2 m} \\ . & . & … & . \\ . & . & … & . \\ . & . & … & . \\ {a}_{k 1} & {a}_{k 2} & … & {a}_{km}\end{matrix} )$$

where k is the number of sequences in the alignment and m≥n is the length of the alignment. Without loss of generality, it is assumed that x is the first sequence in the alignment; that is, there exists an injective map g:[n]→[m] such that i≤g(i) and xᵢ=a_{lg(i)} for all i∈[n].

In one embodiment, the position-specific scoring matrices A may then be passed to the hidden Markov model (HMM) profile generation module 120 that generates HMM profiles from the MSA matrices A. For example, let h:{aᵢⱼ∈A}→+{M, I, D} be the MSA state function which maps amino acids to the three possible states in an MSA:

1. Match: aᵢⱼ is an amino acid that is related, evolutionarily or structurally, to other amino acids in column j.
2. Insertion: aᵢⱼ is an amino acid that is not related to other amino acids in its column but is more likely the result of a mutation that inserted additional amino acids.
3. Deletion: aᵢⱼ is not an amino acid, but rather a column in which protein i is missing an amino acid where other proteins in the MSA have amino acids that are either matched or inserted.

Thus, the HMM profile generation module 120 may build profile HMM from the MSA matrix represented by the match state emissions p₁^{M}, p₂^{M}, . . . , pₗ^{M} and the insertion state emissions p₁^{I}, p₂^{I}, . . . , pₗ^{I} as well as an injective function ƒ:[l]→[m] which maps the indices of the profile back to the columns of the MSA matrix A. pⱼ^{M} and pⱼ^{I} are probability vectors of size S containing the probability of seeing each amino acid in column f(j) in match or insertions states respectively:

$${\sum }_{s = 1}^{S} {( {p}_{j}^{M} )}_{s} = 1 , {( {p}_{j}^{M} )}_{s} \geq 0 and {\sum }_{s = 1}^{S} {( {p}_{j}^{I} )}_{s} = 1 , {( {p}_{j}^{I} )}_{s} \geq 0$$

for an amino acid alphabet of size S. For example, the standard 20 amino acids during profile creation may be used.

In one embodiment, if f has a well-defined inverse f⁻¹: [m]→[l], the HMM profile generation module 120 may generate a sequence of vector labels 125, l₁, l₂ . . . lₙ associated with the input sequence x, defined as:

$${l}_{i} ( x ) = \{ \begin{matrix}{p}_{{f}^{- 1} ( g ( i ) )}^{M} , & if h ( {a}_{1 g ( i )} ) = M \\ {p}_{{f}^{- 1} ( g ( i ) )}^{I} , & if h ( {a}_{1 g ( i )} ) = I\end{matrix}$$

The lᵢ(x) are well-defined: h(a_{1g(i)})≠D, ∀i since g(i) only maps to columns in the alignment where x contains amino acids.

The generated HMM profiles may then be sent to the language model 130, to generate profile prediction probabilities 132 of the input data sequence 102. For example, given a network function F, the output profile prediction of the language model can be represented as F_{i,s}(x; θ), 1≤i≤n, s∈S, where θ represents the parameter of the language model 130.

The HMM profile labels lᵢ(x) 125 may also be sent to the loss module 140, where the loss module 140 compares the HMM profile labels 125 and the profile prediction 132 from the language model 130 to compute the profile prediction loss as:

$${L}_{PP} ( x , θ ) = \frac{1}{n} {\sum }_{i = 1}^{n} {\sum }_{s = 1}^{S} {l}_{i , s} ( x ) ( log ( {l}_{i , s} ( x ) ) - log ( {F}_{i , s} ( x ; θ ) ) ) = \frac{1}{n} {\sum }_{i = 1}^{n} KLDiv ( {l}_{i} ( x ) , {F}_{i} ( x ; θ ) )$$

where for F_{i,s}(x; θ) and lᵢ(x) the i index represents the respective sequence position and the s index represents the respective amino acid output probability. In another embodiment, a masked language modeling objective for the language model 130 may also be computed using the HMM profile labels 125 and the profile prediction outputs 132:

$${L}_{MLM} ( x , θ ) = \frac{1}{n} {\sum }_{i ∈ mask} {\sum }_{s = 1}^{S} {L}_{i , s} ( x ) ( log ( {F}_{i , s} ( x ; θ ) ) = \frac{1}{n} {\sum }_{i ∈ mask} CrossEntropy ( {L}_{i} ( x ) , {F}_{i} ( x ; θ ) )$$

for one-hot labels L_{i,s}(x) that are equal to 1 if xᵢ is the sth amino-acid in the vocabulary, and 0 otherwise; and “mask” denotes a set of indexes where the tokens at such respective position has been masked.

The loss module 140 may then compute a joint loss:

L_{JOINT}(x,θ,λ)=λL_{MLM}(x,θ)+(1−λ)L_{PP}(x,θ)

for a scaling parameter λ. For example, the parameter λ may be empirically set, and/or dynamically adjusted such that L_{MLM}(x, θ)≈L_{PP}(x, θ) during training.

The language model 130 may then be updated by the joint loss via the backpropagation path 150. From an NLP perspective, profile prediction at the language model 130 may be similar to predicting a distribution over possible ways to rephrase a sentence while preserving its meaning from only 1the original sentence itself. This requires not only knowing which words carry the meaning of the sentence but also knowing the synonyms of these words in the context of that sentence, which often entails a significant understanding of language. As such, the language model 130 is pre-trained to learn about the underlying protein biology more than simply predicting masked-out 1amino acids by learning through the joint loss.

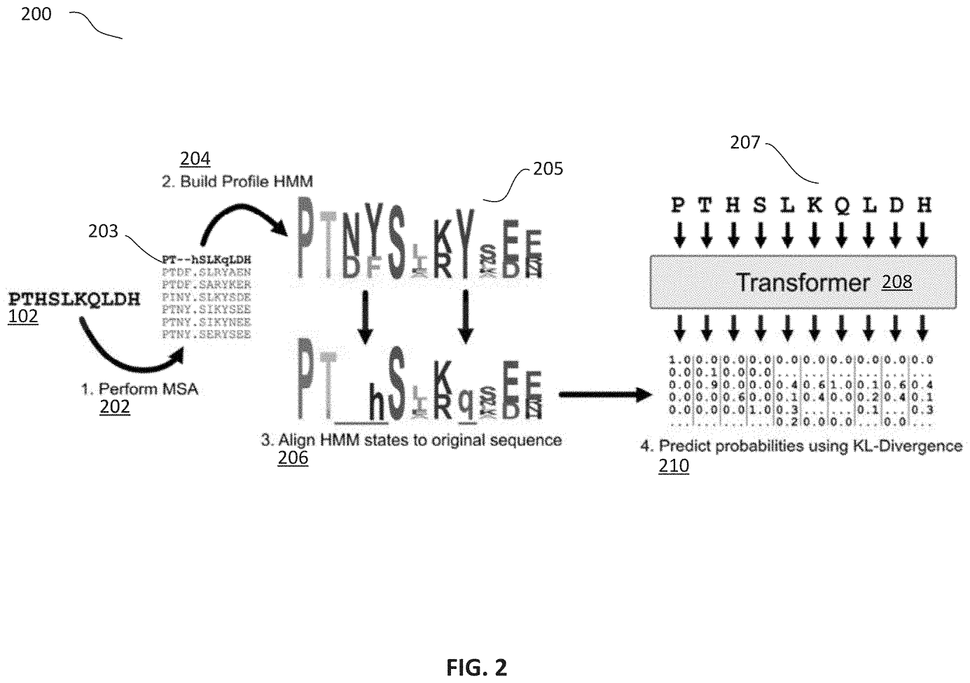

FIG. 2 illustrates an overview process of the proposed task of generating labels from HMM profiles during pre-training, according to embodiments described herein. As shown at step 1 (202), an initial sequence 102 of “PTHSLKQLDH” is retrieved. An MSA matrix 203 for that sequence 102 is generated by searching the sequence against a reference database. At step 2 (204), a profile HMM is generated for the multiple sequence alignment and the HMM states are aligned to the original sequence at step 3 (206). For example, the first H and the Q in the sequence correspond to inserted amino acids that didn't match any columns in the alignment. Therefore, for those amino acids, insertion state emissions are used as labels rather than match state emissions. The rest of the amino acids in the sequence were in match states, so the match state emission probabilities are used as labels. Thus, the protein has deletions in two of the match states in the MSA (columns 2 and 3), which are omitted from the label since they have no corresponding amino acids as inputs. Finally, at step 4 (210), the corresponding label is predicted by the transformer network 208 in response to the input sequence 207. The predicted probabilities 210 is then compared with the computed HMM labels using KL divergence, averaged over the length of the sequence, as the loss objective to train the transformer network 208.

### Computer Environment

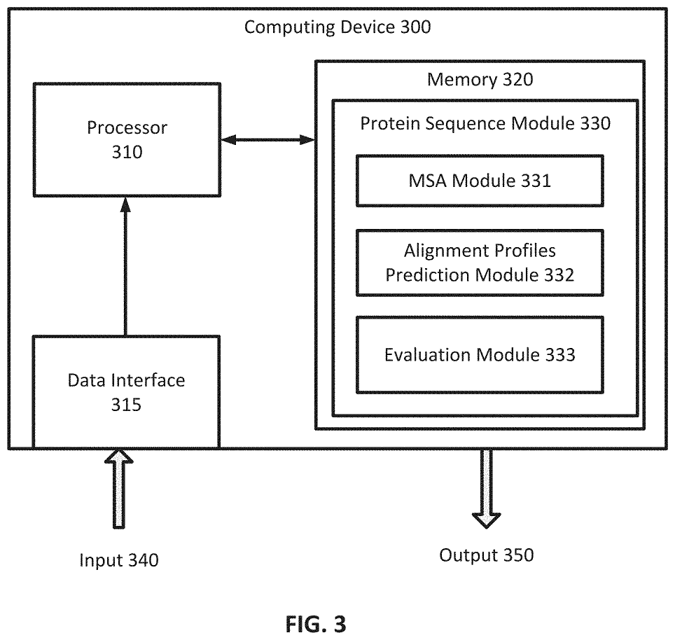

FIG. 3 is a simplified diagram of a computing device 300 that implements and pre-trains a protein sequence model, according to some embodiments. As shown in FIG. 3, computing device 300 includes a processor 310 coupled to memory 320. Operation of computing device 300 is controlled by processor 310. And although computing device 300 is shown with only one processor 310, it is understood that processor 310 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in computing device 300. Computing device 300 may be implemented as a stand-alone subsystem, as a board added to a computing device, and/or as a virtual machine.

Memory 320 may be used to store software executed by computing device 300 and/or one or more data structures used during operation of computing device 300. Memory 320 may include one or more types of machine readable media. Some common forms of machine readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Processor 310 and/or memory 320 may be arranged in any suitable physical arrangement. In some embodiments, processor 310 and/or memory 320 may be implemented on a same board, in a same package (e.g., system-in-package), on a same chip (e.g., system-on-chip), and/or the like. In some embodiments, processor 310 and/or memory 320 may include distributed, virtualized, and/or containerized computing resources. Consistent with such embodiments, processor 310 and/or memory 320 may be located in one or more data centers and/or cloud computing facilities.

As shown, memory 320 includes a protein sequence module 330 that may be used, in some examples, for generative modeling for protein engineering. In some examples, protein sequence module 330 may be implemented using hardware, software, and/or a combination of hardware and software. In some examples, memory 320 may include non-transitory, tangible, machine readable media that includes executable code that when run by one or more processors (e.g., processor 310) may cause the one or more processors to perform the methods described in further detail herein.

As shown, computing device 300 receives input 340, via a data interface 315. For example, the input 340 may include protein sequence data that is loaded from a remote database, and the data interface 315 may include a network interface to receive data including the protein sequence data. The input 340 is provided to protein sequence module 330. This input 350 may comprise data for one or more sequences of amino acids that constitute proteins, and/or the like. Protein sequence module 330 may generate output 350, which may comprise data indicating the structural and/or functional properties of the protein sequences in the input 340.

According to some embodiments, protein sequence module 330 may implement and/or emulate one or more neural network systems and models, and corresponding methods, for modeling for protein engineering. In some embodiments, the neural network model for protein engineering in the protein sequence module 330 may comprise, incorporate, or employ a neural network model that has been developed for natural language processing (NLP).

The protein sequence module 330 may include one or more submodules such as an MSA module 331, an alignment profiles prediction module 332 and an evaluation module 333. The MSA module 331 is configured to arrange proteins in a matrix whose rows are individual protein sequences and whose columns contain amino acids that either come from the same position in some ancestral sequence (homologous), or play a common structural or functional role. For example, pre-training data that comprises of some 32 million sequences from Pfam may be used. Pfam further contains pre-built MSAs for each of its entries, grouped into a set of families.

The alignment profiles prediction module 332 fit a profile HMM underlying the protein sequence. Specifically, the alignment profiles prediction module 332 models the probabilities of amino acids appearing in the columns of an MSA, as well as the probability of inserting additional amino acids between columns or missing existing columns. Features derived from profile HMMs often contain information about the evolutionary history of a protein. In particular, the emission probabilities give insight into which positions in the proteins are likely to mutate or remain constant over the course of evolution. This in turn illuminates which portions of the protein are critical for the protein's structure or function. Thus, profile HMMs are built from multiple sequence alignments using HMMER with the default arguments.

One task of the alignment profiles prediction module 332 is to predict a protein's profile HMM directly from it sequence. There are three cases to handle when turning a profile HMM into a label. Considering an input protein's amino acids one at a time, the first case is if an amino acid in a protein sequence corresponds to a match state in the profile. In this case the profile's match state emission probabilities at that amino acid's column is used as the label. This represents a distribution over amino acids occurring at this column across the MSA. The second case is if an amino acid in a protein sequence corresponds to an insertion state in the profile. In this case, the insertion state emission probabilities are used at that column as the label. This represents a distribution of amino acids that have been inserted before this column across the MSA. The third case is if a protein sequence is missing an amino acid in a match column of the MSA. In this case any input or target label at this column may be omitted. Further description of this process can be described in relation to FIGS. 1-2.

Thus, the alignment profiles prediction module 332 generates a label representing a probability distribution for each input amino acid. The final loss function is the KL divergence between the label and the transformer's output after passing it through the softmax function. This loss function is averaged over the length of the sequence.

The alignment profiles prediction task at module 332 is akin to predicting a distribution over possible ways to rephrase a sentence while preserving its meaning from only the original sentence itself. This requires not only knowing which words carry the meaning of the sentence but also knowing the synonyms of these words in the context of that sentence. Doing so would require a significant understanding of language. As such, the alignment profiles prediction module 332 encourages the neural network to learn about underlying protein biology more than simply predicting masked-out amino acids.

The evaluation module 334 is configured to evaluate the pre-training task using a set of five standardized protein sequence prediction tasks with associated datasets plus a large unlabeled pre-training dataset derived from Pfam. For example, labels for the pre-training data set may be produced by submodules 331-333, and then the pre-trained models can be evaluated based on several downstream tasks, such as but not limited to secondary structure prediction, contact prediction, fluorescence prediction and stability prediction, and/or the like.

The protein sequence module 330, and/or the submodules 331-333 may be implemented via software, hardware, or a combination thereof.

### Profile Prediction Work Flow

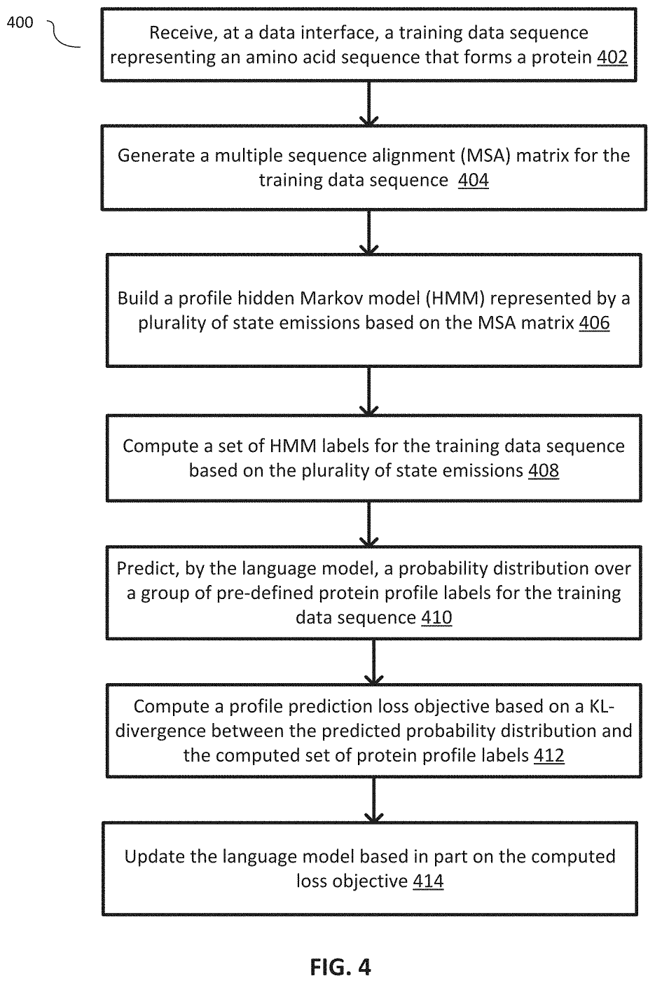

FIG. 4 is a simplified logic flow diagram illustrating a method for pre-training a transformer network for protein profile prediction, according to some embodiments described herein. One or more of the processes 402-410 of method 400 may be implemented, at least in part, in the form of executable code stored on non-transitory, tangible, machine-readable media that when run by one or more processors may cause the one or more processors to perform one or more of the processes 402-410. In some embodiments, method 400 may correspond to the method used by the module 330.

At step 402, a training data sequence (e.g., input sequence 102) representing an amino acid sequence that forms a protein may be received at a data interface.

At step 404, an MSA matrix may be generated for the training data sequence. For example, the MSA matrix A is generated by searching the training data sequence against a reference database of data sequences representing different proteins. In this way, the MSA matrix A is formed with rows representing individual protein sequences and columns representing amino acids that either come from a same position in an ancestral sequence or play a common structural or functional role.

At step 406, a profile hidden Markov model (HMM) may be built. The profile HMM model may be characterized by a plurality of state emissions based on the MSA matrix. For example, the profile HMM is built by a MSA state function that maps each entry of the MSA matrix to any of a MSA match state emission, a MSA insertion state emission and a MSA deletion state emission. Further details of the state emissions may be found in relation to FIG. 1.

At step 408, a set of HMM labels are computed for the training data sequence based on the plurality of state emissions. For example, one or more HMM states of the profile HMM are aligned to one or more tokens in the training data sequence, and then it is determined whether to use a corresponding MSA insertion state emission or a corresponding MSA match state emission as a HMM label based on the alignment, as described in relation to FIG. 1.

At step 410, the language model predicts a probability distribution over a group of pre-defined protein profile labels for the training data sequence.

At step 412, a profile prediction loss objective L_{PP} (x, θ) is computed based on a KL-divergence between the predicted probability distribution and the computed set of protein profile labels. In another implementation, the training data sequence may be perturbed with one or more mask tokens such that the language model predicts a masked output probability distribution over the group of pre-defined protein profile labels for the perturbed training data sequence. A masked learning loss objective is computed based on a cross entropy between the predicted masked output probability distribution and one-hot labels of the training data sequence. The one-hot labels of the training data sequence are defined based on whether a respective token in the training data sequence corresponds to a certain amino acid in a protein vocabulary.

At step 414, the language model may be updated based in part on the computed profile prediction loss objective. In one implementation, a weighted sum of the profile prediction loss objective and the masked learning loss objective may be computed, which may be used to update the language model based on the weighted sum.

In one embodiment, steps 410-414 may be repeated during a training epoch to iterate all training sequences in the training dataset.

### Example Performance

In some embodiments, the language model pre-trained using the procedure described herein is evaluated using the TAPE benchmark: a set of five standardized protein sequence prediction tasks with associated datasets plus a large unlabeled pre-training dataset derived from Pfam. Labels may be built for the pre-training data set using the procedure described in FIGS. 1-4. The pre-trained models are then evaluated on the five downstream TAPE tasks: the secondary structure prediction described in Klausen et al., Netsurfp-2.0: Improved prediction of protein struc-tural features by integrated deep learning. Proteins: Structure, Function, and Bioinformatics, 87(6):520-527, 2019, the contact prediction described in AlQuraishi, Proteinnet: a standardized data set for machine learning of protein structure. BMC bioinformatics, 20(1):1-10, 2019, remote homology detection described in Hou et al., Deepsf: deep convolutional neural network for mapping protein sequences to folds, Bioinformatics, 34(8):1295-1303, 2018, fluorescence prediction described in Sarkisyan et al., Local fitness landscape of the green fluorescent protein. Nature, 533(7603):397-401, 2016, and stability prediction described in Rocklin et al., Global analysis of protein folding using massively parallel design, synthesis, and testing. Science, 357(6347):168-175, 2017, using the metrics specified by TAPE.

In some embodiments, a transformer architecture used by Rao et al. can be pre-trained by the profile prediction pre-training embodiment discussed herein, but the pre-training task is not architecture-specific and may be applied to a generic neural network.

For example, three models with the three different objectives L_{PP}(x, θ), L_{MLM}(x, θ), L_{JOINT}(x, θ). The profile prediction model used a learning rate of 0.00025, while the multi-task and masked language modeling models use a learning rate of 0.0001. These learning rates represented the largest learning rates that did not cause the model to diverge during the course of training, searching from 0.00001 in increments of 0.00005. All models were pre-trained for 34 epochs. The learning rate uses a warm-up schedule and dynamic batch sizing, both of which are described in Rao et al. Pre-training a single model may take approximately two weeks with processor 310, which may comprise 8 NVIDIA Tesla V100 GPUs.

Training details for all downstream tasks can be similar to the procedure laid out by Rao et al.: for example, a learning rate of 0.0001 with linear warm-up schedule, the Adam optimizer and backpropagation through the entire pre-trained model. The downstream prediction heads all follow those in Rao et al., except for contact prediction which uses a single linear layer rather than a 30-layer convolutional architecture.

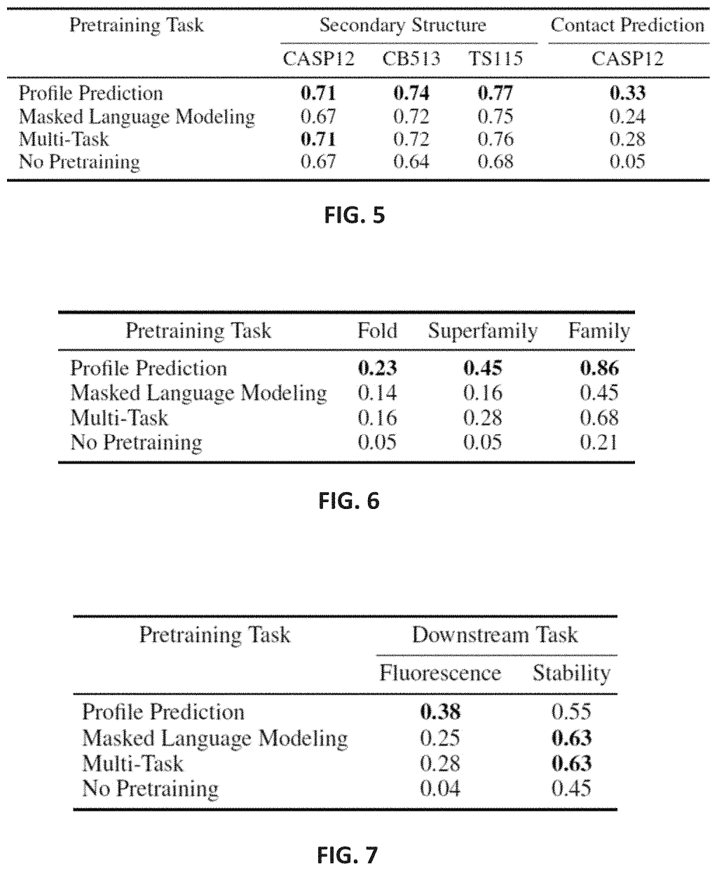

The pre-training task described in FIGS. 1-4 is compared against masked language modeling and the multitask model which combines both tasks, keeping hyperparameters and architecture fixed. The results are shown in FIGS. 5-7. For both structure prediction tasks—secondary structure and contact prediction—profile prediction pre-training outperforms multitasking, which in turn outperforms masked language modeling. All three tasks outperform the same model that was not pre-trained. Although it is not surprising that profile pre-training outperforms mask language modeling on structure prediction—namely because HMM profiles are known to contain information relevant to a protein's structure—the differences between the evaluated models are not large. This may mean that potentially more than just a new pre-training task is needed to continue to improve structure predictors, such as different architectures, or larger pre-training datasets.

The remote homology detection task demonstrates the largest gap between profile prediction and mask language modeling. The model pre-trained with profile prediction is about 2 to 3 times more accurate than the model pre-trained using masked language modeling. The performance of the multitask model lies between that of the other two models and all three again outperform a randomly initialized model. This may be because HMM profiles also contain significant amounts of information about evolutionarily related proteins, which is closely related to the structural or functional groupings that a protein falls into.

The same pattern is observed on the fluorescence task: profile prediction leads to the best test set performance, followed by multitasking, masked language modeling and no pre-training in that order. Finally, on the stability task, the masked language modeling model and the multitask model both outperform profile prediction. This may be because this task tests models' ability to generalize to proteins with a single amino acid difference from proteins in the training set—a task that masked language modeling is particularly suited for. Taken as a whole, these results indicate that there may not be a one-size-fits-all pre-training task for all downstream prediction tasks. Rather, it may be beneficial to tailor the pre-training task to the downstream task: for structure or evolutionary tasks, incorporating profile information may be beneficial, but for fine-grained engineering tasks, masked language modeling may be a better choice.

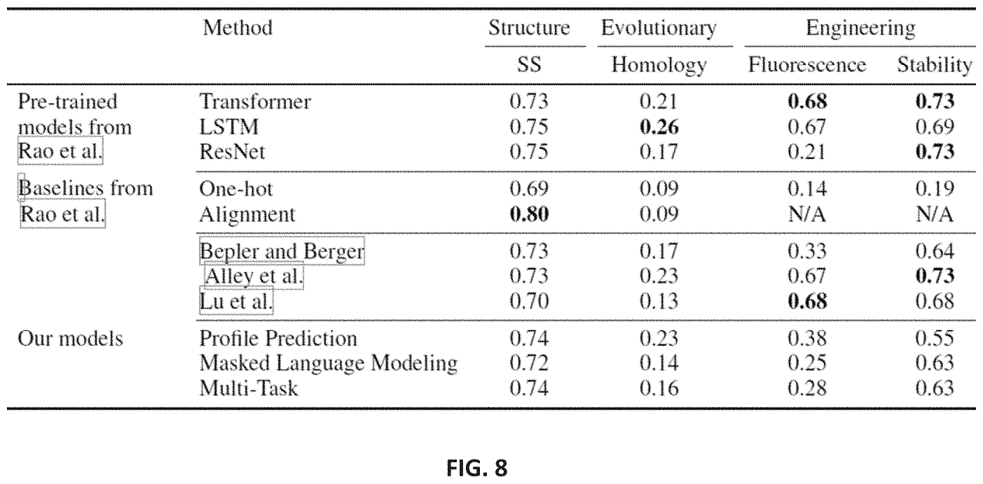

The pre-training task described herein is also compared against the models presented in the original TAPE benchmark, as well as some existing pre-training method that makes use of the TAPE benchmark. For secondary structure task, results are presented from the CB513 test set. For remote homology detection results are presented from the fold level prediction task. The results are presented in FIG. 8.

On the secondary structure task, the propose pre-training task is compared against the NetsurfP2.0 model presented by Klausen et al., Netsurfp-2.0: Improved prediction of protein struc-tural features by integrated deep learning, Proteins: Structure, Function, and Bioinformatics, 87(6):520-527, 2019, which is hereby expressly incorporated by reference herein in its entirety, and is the alignment baseline from Rao et al. The propose pre-training task is also compared against LSTM 185 and ResNet models from Rao et al., and outperforms both the Transformer model as well as all previous work proposing protein-specific pre-training tasks as described in Bepler et al., Learning protein sequence embeddings using information from structure, in Proceedings of International Conference on Learning Representations, 2018 and Lu et al., Self-supervised contrastive learning of protein representations by mutual information maximization, bioRxiv 2020), and the auto-regressive LSTM in Alley et al., Unified rational protein engineering with sequence-only deep representation learning, bioRxiv, page 589333, 2019. On the remote homology task, the pre-training task outperforms all existing models except the TAPE benchmark's LSTM model and the LSTM presented by Alley et al. It is again noted that the pre-training task outperforms the protein-specific pre-training tasks in Bepler et al. and Lu et al.

It is worth noting that the pre-training task described in FIGS. 1-4 are not mutually exclusive with existing pre-training methods. In one embodiment, the pre-training task described in FIGS. 1-4 may be combined with the architectures and pre-training tasks present in existing work to pre-train a language model, or any other neural network.

One or more of the processes shown in FIGS. 1-4 may be implemented, at least in part, in the form of executable code stored on non-transitory, tangible, machine-readable media that when run by one or more processors may cause the one or more processors to perform one or more of the processes. In some embodiments, the process corresponds to the operation of protein sequence module 130 in FIG. 1.

Some examples of computing devices, such as computing device 100 may include non-transitory, tangible, machine readable media that include executable code that when run by one or more processors (e.g., processor 110) may cause the one or more processors to perform the processes of method 300. Some common forms of machine readable media that may include the processes of method 300 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

This description and the accompanying drawings that illustrate inventive aspects, embodiments, implementations, or applications should not be taken as limiting. Various mechanical, compositional, structural, electrical, and operational changes may be made without departing from the spirit and scope of this description and the claims. In some instances, well-known circuits, structures, or techniques have not been shown or described in detail in order not to obscure the embodiments of this disclosure Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and in a manner consistent with the scope of the embodiments disclosed herein.

What is claimed is:

1. A method for pre-training a language model for protein profile prediction, the method comprising:
receiving, at a data interface, a training data sequence representing an amino acid sequence that forms a protein;
generating a multiple sequence alignment (MSA) matrix for the training data sequence;
building a profile hidden Markov model (HMM) represented by a plurality of state emissions based on the MSA matrix;
computing a set of HMM labels for the training data sequence based on the plurality of state emissions;
predicting, by the language model, a probability distribution over a group of pre-defined protein profile labels for the training data sequence;
computing a profile prediction loss objective based on a KL-divergence between the predicted probability distribution and the computed set of protein profile labels; and
updating the language model based in part on the computed profile prediction loss objective.

2. The method of claim 1, wherein the MSA matrix is generated by:
searching the training data sequence against a reference database of data sequences representing different proteins; and
forming the MSA matrix of rows representing individual protein sequences and columns representing amino acids that either come from a same position in an ancestral sequence or play a common structural or functional role.

3. The method of claim 1, wherein the profile HMM is built by a MSA state function that maps each entry of the MSA matrix to any of a MSA match state emission, a MSA insertion state emission and a MSA deletion state emission.

4. The method of claim 3, wherein the MSA match state emission corresponds to a state where the respective entry of the MSA matrix represents an amino acid that is related to other amino acids in a same column.

5. The method of claim 3, wherein the MSA insertion state emission corresponds to a state where the respective entry of the MSA matrix represents an amino acid that is related to no other amino acid in a same column and is a mutation that inserted additional amino acids.

6. The method of claim 3, wherein the MSA deletion state emission corresponds to a state where the respective entry of the MSA matrix does not represent an amino acid but a column in which a corresponding protein is missing a certain amino acid.

7. The method of claim 3, further comprising:
aligning one or more HMM states of the profile HMM to one or more tokens in the training data sequence; and
determining whether to use a corresponding MSA insertion state emission or a corresponding MSA match state emission as a HMM label based on the alignment.

8. The method of claim 1, further comprising:
perturbing the training data sequence with one or more mask tokens;
predicting, by the language model, a masked output probability distribution over the group of pre-defined protein profile labels for the perturbed training data sequence;
computing a masked learning loss objective based on a cross entropy between the predicted masked output probability distribution and one-hot labels of the training data sequence,
wherein the one-hot labels of the training data sequence are defined based on whether a respective token in the training data sequence corresponds to a certain amino acid in a protein vocabulary.

9. The method of claim 5, further comprising:
computing a weighted sum of the profile prediction loss objective and the masked learning loss objective; and
updating the language model based on the weighted sum.

10. The method of claim 1, wherein the language model is implemented via a Transformer network.

11. A system for pre-training a language model for protein profile prediction, the system comprising:
a memory that stores the language model;
a data interface that receives a training data sequence representing an amino acid sequence that forms a protein; and
a processor that reads and executes instructions from the memory to perform operations comprising:
generating a multiple sequence alignment (MSA) matrix for the training data sequence;
building a profile hidden Markov model (HMM) represented by a plurality of state emissions based on the MSA matrix;
computing a set of HMM labels for the training data sequence based on the plurality of state emissions;
predicting, by the language model, a probability distribution over a group of pre-defined protein profile labels for the training data sequence;
computing a profile prediction loss objective based on a KL-divergence between the predicted probability distribution and the computed set of protein profile labels; and
updating the language model based in part on the computed profile prediction loss objective.

12. The system of claim 11, wherein the MSA matrix is generated by:
searching the training data sequence against a reference database of data sequences representing different proteins; and
forming the MSA matrix of rows representing individual protein sequences and columns representing amino acids that either come from a same position in an ancestral sequence or play a common structural or functional role.

13. The system of claim 11, wherein the profile HMM is built by a MSA state function that maps each entry of the MSA matrix to any of a MSA match state emission, a MSA insertion state emission and a MSA deletion state emission.

14. The system of claim 13, wherein the MSA match state emission corresponds to a state where the respective entry of the MSA matrix represents an amino acid that is related to other amino acids in a same column.

15. The system of claim 13, wherein the MSA insertion state emission corresponds to a state where the respective entry of the MSA matrix represents an amino acid that is related to no other amino acid in a same column and is a mutation that inserted additional amino acids.

16. The system of claim 13, wherein the MSA deletion state emission corresponds to a state where the respective entry of the MSA matrix does not represent an amino acid but a column in which a corresponding protein is missing a certain amino acid.

17. The system of claim 13, wherein the operations further comprise:
aligning one or more HMM states of the profile HMM to one or more tokens in the training data sequence; and
determining whether to use a corresponding MSA insertion state emission or a corresponding MSA match state emission as a HMM label based on the alignment.

18. The system of claim 11, wherein the operations further comprise:
perturbing the training data sequence with one or more mask tokens;
predicting, by the language model, a masked output probability distribution over the group of pre-defined protein profile labels for the perturbed training data sequence;
computing a masked learning loss objective based on a cross entropy between the predicted masked output probability distribution and one-hot labels of the training data sequence,
wherein the one-hot labels of the training data sequence are defined based on whether a respective token in the training data sequence corresponds to a certain amino acid in a protein vocabulary.

19. The system of claim 15, wherein the operations further comprise:
computing a weighted sum of the profile prediction loss objective and the masked learning loss objective; and
updating the language model based on the weighted sum.

20. A processor-readable non-transitory storage medium storing a plurality of processor-executable instructions for pre-training a language model for protein profile prediction, the instructions executed by a processor to perform operations comprising:
receiving a training data sequence representing an amino acid sequence that forms a protein;
generating a multiple sequence alignment (MSA) matrix for the training data sequence;
building a profile hidden Markov model (HMM) represented by a plurality of state emissions based on the MSA matrix;
computing a set of HMM labels for the training data sequence based on the plurality of state emissions;
predicting, by the language model, a probability distribution over a group of pre-defined protein profile labels for the training data sequence;
computing a profile prediction loss objective based on a KL-divergence between the predicted probability distribution and the computed set of protein profile labels; and
updating the language model based in part on the computed profile prediction loss objective.
